(19) United States
(12) Patent Application Publication
(10) Pub. No.: US 2018/0125096 A1
(43) Pub. Date: May 10, 2018

(54) BIRD-REPELLANT ANIMAL FEED COMPOSITIONS AND METHODS OF PREPARATION THEREOF

(71) Applicants: The United States of America, as Represented by the Secretary of Agriculture, Washington, DC (US); Arkion Life Sciences, LLC, New Castle, DE (US)

(72) Inventors: Kenneth E. Ballinger, JR., Kennett Square, PA (US); James C. Carlson, Fort Collins, CO (US); Scott J. Werner, Wellington, CO (US)

(73) Assignees: The United States of America, as represented by the Secretary of Agriculture, Washington, DC (US); Arkion Life Sciences, LLC, New Castle, DE (US)

(21) Appl. No.: 15/809,547

(22) Filed: Nov. 10, 2017

Related U.S. Application Data
(60) Provisional application No. 62/420,273, filed on Nov. 10, 2016.

(51) Int. Cl. A23K 50/10; A23K 50/20; A23K 10/30; A23K 40/00; A23K 20/10; A61K 39/395; A61K 9/00

(52) U.S. Cl. CPC A23K 50/10; A23K 50/20; A23K 10/30; A61K 2039/54; A23K 20/10; A61K 39/395; A61K 9/0056; A23K 40/00

(57) ABSTRACT

In one aspect, the present invention is directed to an animal feed composition that is resistant to feeding by birds, the composition comprising a feed pellet and one or more additional animal feed ingredients that is not pelletized. The feed pellet has a higher lipid content (w/w) and a higher starch content (w/w) than each of the one or more additional animal feed ingredients that is not pelletized. The invention also relates to methods for preparing and using the animal feed compositions.

### RELATED APPLICATIONS

This application claims priority to U.S. Provisional Patent Application No. 62/420,273 filed on Nov. 10, 2016, the contents of which are incorporated herein in their entirety.

### RELATED FEDERALLY SPONSORED RESEARCH

The work described in this application was sponsored by the United States Department of Agriculture's National Wildlife Research Center in Fort Collins, Colo.

### FIELD OF THE INVENTION

In one aspect, the present invention generally relates to animal feed compositions that are resistant to feeding by birds, and methods of preparing the compositions.

### BACKGROUND OF THE INVENTION

Multiple species of birds seasonally congregate in large roosting groups and exploit the abundant and nutritious food sources found on dairies and feedlots (Besser et al., 1968, Journal of Wildlife Management 32: 179-180; Dolbeer et al., 1978, Wilson Bull. 90: 31-44; LeJeune et al., 2008; Proc. 23^{rd} Vertebr. Pest Conf., San Diego, Calif., page 31; Depenbusch et al., 2011, Human-Wildlife Interactions 6: 64-71). European starlings are especially destructive and have been documented causing damage and consuming livestock feed in animal agricultural operations in the United States, Europe and Australia (Feare, 1992, Proc. 15^{th} Vertebr. Pest Contf., Sacramento, Calif., page 83; Bentz et al., 2007. Managing Vertebrate Invasive Species: Proceedings of an International Symposium, USIDA/APHIS/WS, National Wildlife Research Center, Fort Collins, Colo., pages 361-364; Carlson et al., 2011, J. Appl. Ecol. 2: 479-486). In the United States, starlings will congregate with blackbird species to form large foraging and roosting flocks during the winter.

Livestock feed consumption by birds appears to cause economically significant damage to feedlots and dairies in the United States (Glahn and Otis, 1981, ASTM Special Technical Publication No. 752; Twedt and Glahn, 1982, Proc. 10^{th} Vertebr. Pest Conf. Monterey, Calif., pages 159-163). Depenbusch et al. (cited above) estimated that starling consumption of finishing rations in a Kansas feedlot increased operating costs by $0.92/head/day. Estimates of bird damage in commercial dairies within Wisconsin, New York and Pennsylvania suggest that starling damage resulted in $64,000 of feed loss annually within dairies experiencing 10,000 or more birds per day and feed costs per hundred pounds of milk produced (cwt) increased 42% in dairies with ≥10,000 or more birds (Shwiff et al., 2012, J. Dairy Sci. 95: 6820-6829).

Consumption of livestock feed by birds is also associated with the microbiological contamination of feedlots and dairies including antimicrobial resistant bacteria (LeJeune et al. 2001, Journal of Dairy Science, 84, 1856-1862; Carlson et al., 2011. Journal of Dairy Science, 84, 1856-1862; Carlson et al., 2011, BMC Veterinary Research 7, 9; Carlson et al., 2015, Vet. Microbiol. 179: 60-68). Additionally, reducing starling numbers on feedlots was shown to reduce Salmonella enterica contamination within cattle feed and water supplies (Carlson et al., 2011, BMC Veterinary Research 7, 9). A genetic analysis of the S. enterica isolates recovered from starlings, cattle, cattle feed and cattle water troughs showed that bacteria was being transmitted between starlings and cattle and that their shared feed sources were the likely conduit. This information suggests bird consumption of livestock rations leads to the amplification and spread of microorganisms of concern to public health, including antimicrobial resistant bacteria, in feedlots and dairies. The economic impacts of disease transmission likely eclipse those of feed depredation but they have not been estimated.

Livestock feed consumption by birds appears to impact animal performance in feedlots and dairies. For example. Wright (1973, EPPO Bulletin 2: 85-89) and Feare and Swannack (1978, Animal Prod. 26: 259-265) found increased weight gain in cattle when fed in bird-excluded areas. Feare (1984, The Starling, Oxford University Press, Oxford, New York, USA) suggested if feed consumption by birds occurs at the bunk then removal of high-energy feed ingredients may reduce animal performance and these losses may be economically significant to producers. Depenbusch et al. (2011, cited above) provided nutritional comparisons of cattle rations before and after starling damage and concluded these changes could decrease growth rates and feed conversion efficiency of feeder cattle.

In conclusion, bird feeding in animal agriculture facilities causes direct economic losses to producers, lost production and increased risk of microbial contamination of livestock feed and water supplies. All three of these forms of damage are directly attributable to bird consumption of livestock feed. Consequently, addressing bird damage in animal agricultural operations can be most effectively accomplished by protecting or excluding livestock feed from birds. Thus, a need exists for animal feeds that reduce or eliminate consumption by birds.

### SUMMARY OF THE INVENTION

In certain aspects, the invention relates to an animal feed composition that is resistant to feeding by birds, the composition comprising: an animal feed pellet having a diameter greater than or equal to 9.5 mm; and one or more additional animal feed ingredients, wherein the animal feed pellet has a higher lipid content (w/w) and/or a higher starch content (w/w) than each of the one or more additional animal feed ingredients. In certain embodiments, the one or more additional animal feed ingredients have a diameter less than 9.5 mm. In certain embodiments, the one or more additional animal feed ingredients are not pelletized. In certain embodiments, the animal feed pellet comprises an animal feed ingredient selected from the group consisting of steam flaked corn, cracked corn and ground corn. In certain embodiments, the animal feed pellet comprises at least 30% w/w of starch. In certain embodiments, the animal feed pellet comprises at least 5% w/w of fat. In certain embodiments, the animal is a ruminant animal, poultry or swine. In certain embodiments, the composition further comprises an effective amount of a compound selected from the group consisting of a bird repellant, a rodent repellant, and a wildlife attractant. In certain embodiments, the composition further comprises an antibody.

In certain aspects, the invention relates to a method of preparing an animal feed composition that is resistant to feeding by birds, the method comprising: (a) determining fat content and starch content in at least two animal feed ingredients; (b) selecting one or more animal feed ingredients based on a parameter selected from the group consisting of: (i) a fat content greater than or equal to a predetermined value for fat content; and (ii) a starch content greater than or equal to a predetermined value for starch content; (c) forming an animal feed pellet comprising the animal feed ingredients having a fat content or a starch content greater than or equal to the predetermined value; and (d) mixing the animal feed pellet with one or more animal feed ingredients that have a fat content and a starch content less than the predetermined value, thereby preparing the animal feed composition, wherein the one or more animal feed ingredients that have a fat content and a starch content less than the predetermined value are not pelletized. In certain embodiments, the predetermined value for starch content is 30% w/w. In certain embodiments, the predetermined value for fat content is 5% w/w. In certain embodiments, the animal feed pellet has a diameter greater than or equal to 9.5 mm. In certain embodiments, the animal feed pellet comprises an animal feed ingredient selected from the group consisting of steam flaked corn, cracked corn and ground corn. In certain embodiments, the animal is a ruminant animal, poultry or swine. In certain embodiments, the method further comprises adding an effective amount of a compound selected from the group consisting of a bird repellant, a rodent repellant and a wildlife attractant to the animal feed composition. In certain embodiments, the method further comprises adding an antibody to the animal feed composition.

In certain aspects, the invention relates to an animal feed pellet comprising at least 5% w/w of fat and at least 30% w/w of starch and having a diameter greater than or equal to 9.5 mm. In certain embodiments, the pellet comprises a total mixed ration (TMR). In certain embodiments, the pellet comprises an animal feed ingredient selected from the group consisting of steam flaked corn, cracked corn and ground corn. In certain embodiments, the animal is a ruminant animal, poultry or swine. In certain embodiments, the pellet comprises an effective amount of a compound selected from the group consisting of a bird repellant and a rodent repellant. In certain embodiments, the pellet further comprises an antibody.

In certain aspects, the invention relates to a method of feeding an animal, comprising administering an aforementioned animal feed composition to an animal.

In certain aspects the invention relates to a method of feeding an animal, comprising administering an aforementioned animal feed pellet to an animal.

In certain aspects, the invention relates to a method of feeding an animal, comprising administering an animal feed composition or animal feed pellet as described herein.

In certain aspects, the invention relates to a method of increasing weight gain in an animal, comprising administering an animal feed composition or animal feed pellet as described herein to an animal.

In certain aspects, the invention relates to a method of increasing milk production in a dairy cow, comprising administering an animal feed composition or an animal feed pellet as described herein to a dairy cow.

In certain embodiments of the aforementioned methods, the animal feed composition or the animal feed pellet is exposed to predation by birds during consumption of the feed by the animal or the dairy cow. In certain embodiments of the aforementioned methods, the animal is a ruminant animal, poultry or swine.

### BRIEF DESCRIPTION OF THE FIGURES

FIG. 1 shows animal feed component selection by Red-winged blackbirds for dairy total mixed rations (TMR).

FIG. 2 shows animal feed component selection by European starlings for dairy total mixed rations (TMR) with fat nugget. SFC refers to steam flaked corn; DDG refers to dried distillers grain.

FIG. 3 shows animal feed component selection by European starlings for dairy total mixed rations (TMR) without energy nugget. SFC refers to steam flaked corn; DDG refers to dried distillers grain.

FIG. 4 shows consumption of poultry feed pellets of various sizes by European starlings.

### DETAILED DESCRIPTION OF THE INVENTION

In certain aspects, the present invention relates to an animal feed composition that is resistant to feeding by birds, the composition comprising: (a) a feed pellet; and (b) one or more additional animal feed ingredients that are not pelletized, wherein the feed pellet has a higher lipid content (w/w) and a higher starch content (w/w) than each of the one or more additional animal feed ingredients that is not pelletized. Applicants have demonstrated through controlled feeding studies with Red-winged blackbirds and European starlings that these wild bird species prefer animal feed ingredients with high starch and/or fat content. In addition, Applicants have demonstrated that consumption of feed pellets by European starlings was effectively inhibited at feed pellet sizes of about 9.5 mm or greater. Accordingly, consumption of animal feeds by birds may be reduced by incorporating the animal feed ingredients with high starch and/or fat content into a pellet that is of sufficient size to deter bird feeding.

### 1. Animal Feed Ingredients and Animal Feed Compositions

The term “animal feed ingredient” refers to any compound or composition that is used to nourish an animal. Exemplary animal feed ingredients include, for example, grains and grain products, other plant products such as hay, animal products, vitamin supplements, mineral supplements, and mixtures thereof. Particular examples of animal feed ingredients include, but are not limited to, oilseed byproducts (e.g. soybean meal, canola meal and whole cottonseed), alfalfa hay, corn silage, grass hay, ground corn, lactating mineral, steam flaked corn, wet brewers grain, and wet distillers grain. In a particular embodiment, an animal feed ingredient can be an animal feed pellet that is high in fat, e.g. a PROPEL® energy nugget (Purina Mills, St. Louis, Mo.). Animal feed ingredients are known in the art and are described, for example, in US 2007/0172540. Additional examples of animal feed ingredients are provided below.

The term “animal feed composition” refers to a composition comprising one or more animal feed ingredients. The one or more animal feed ingredients can be combined to form the animal feed composition. In certain embodiments, the animal feed composition is prepared as a total mixed ration (TMR). A TMR is composed of a combination of one or more animal feed ingredients such as forages, and any additional animal feed ingredients such as grains, protein supplement(s), minerals, and vitamins that may be needed. The animal feed ingredients can be mixed together to form a TMR having a balanced ration in which the weight of each ingredient can be selected and controlled. Such a TMR may then be offered to cattle or other livestock as their sole source of feed.

In certain aspects, the invention is directed to an animal feed composition that is resistant to feeding by birds. In certain embodiments, the animal feed composition is prepared in the form of a bird resistant animal feed pellet. One method of preventing feeding by birds is to adjust the size of the an animal feed pellet such that it is too large or too small to be consumed by birds. For example, in some embodiments, the size of the bird resistant animal feed pellet is optimized such that the pellet is large enough to reduce feeding by birds, but small enough to prevent selective feeding by the animal to which the feed is administered. For example, in some embodiments, the animal feed pellet diameter is greater than 6, 6.5, 7, 7.5, 8, 8.5, 9, 9.5, 10, 10.5, 11, 11.5, 12, 12.5, 13, 13.5 14, 14.5, 15, 16, 17, 18, 19, 20, 21, 22, 23, 24, 25, 26, 27, 28, 29, 30, 40, or 50 mm. In some embodiments the animal feed pellet diameter is less than 11, 11.5, 12, 12.5, 13, 13.5 14, 14.5, 15, 16, 17, 18, 19, 20, 21, 22, 23, 24, 25, 26, 27, 28, 29, 30, 40, or 50 mm. Any of these values may be used to define a range for the size of the pellet. For example, in some embodiments, the pellet diameter is 9.5-25 mm, 10-20 mm, or 10-25 mm. In some embodiments, the size of the animal feed pellet is optimized to produce a pellet that is too small to be consumed by birds. For example, in some embodiments, the diameter of the animal feed pellet is less than 1 mm. In some embodiments, the animal feed pellet comprises 2, 3, 4, 5 or more animal feed ingredients.

It is further contemplated that the animal feed composition consists of one or more bird resistant animal feed pellets that comprise the entire animal feed composition. Accordingly, in some embodiments, the present invention relates to one or more bird resistant animal feed pellets comprising a TMR. In a particular embodiment, the bird resistant animal feed pellet consists of a TMR. It is preferred that the bird resistant pellet be optimized such that the pellet is large enough to reduce feeding by birds, but small enough to prevent selective feeding by the animal to which the feed is administered.

Alternatively, the animal feed composition comprises one or more bird resistant animal feed pellets and one or more additional animal feed ingredients that are not pelletized, wherein the bird resistant animal feed pellet have a higher fat content (w/w) and/or a higher starch content (w/w) than each of the one or more additional animal feed ingredients that are not pelletized. In some embodiments, the animal feed composition is a TMR comprising both bird resistant pellets and non-pelletized animal feed ingredients. The bird resistant animal feed pellets may be optimized such that the pellets are large enough to reduce feeding by birds, but small enough to prevent selective feeding by the animal to which the feed is administered, and contain the animal feed ingredients that are high in starch and/or fat, while the remaining animal feed ingredients are either not pelletized or contained in pellets that are not large enough to reduce feeding by birds.

The animal feed composition may comprise a “nutritionally effective amount” of an animal feed ingredient. A nutritionally effective amount of an animal feed ingredient is that amount which will provide the protein, fat, carbohydrate, vitamin, supplement, caloric or energy value, or other nutritional value desired for the particular animal feed ingredient. The animal feed composition may comprise at least 0.1%, 0.5%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75% or 80% w/w of an animal feed ingredient, such as an animal feed ingredient described above. In some embodiments, the animal feed composition comprises less than 0.5%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75% or 80% w/w of an animal feed ingredient, such as an animal feed ingredient described above. Any of these values may be used to define a range for the percentage by weight of the animal feed ingredient in the animal feed composition. For example, the animal feed composition may comprise from 0.1% to 1% w/w, from 1% to 5% w/w, or from 1% to 10% w/w of an animal feed ingredient.

In a particular embodiment, the animal feed composition is a TMR comprising alfalfa hay, canola meal, corn silage, an energy nugget, grass hay, ground corn, lactating mineral, soybean meal, steam flaked corn, wet brewers grain, wet distillers grain, and whole cottonseed. In a further particular embodiment, the animal feed composition is a TMR comprising about 9.9% w/w alfalfa hay, about 6.5% w/w canola meal, about 29.5% w/w corn silage, about 1.7% w/w energy nugget, about 2.9% w/w grass hay, about 12.6% w/w ground corn, about 3.4% w/w lactating mineral, about 6.7% w/w soybean meal, about 13.8% w/w steam flaked corn, about 3.2% w/w wet brewers grain, about 3.1% w/w wet distillers grain, and about 6.7% w/w whole cottonseed.

Animal Feed Ingredients

Oilseed Byproducts:

These byproducts are the materials left after the vegetable oil content has been extracted from the oilseeds. These byproducts include: soybean meals (high and low protein grade), soybean hulls, whole cottonseed, cottonseed meal, cottonseed hulls, canola meal, sunflower seed meal, linseed meal, corn meal, rapeseed meal and safflower meal. Other materials from which oils are commonly extracted may also be included in this group, e.g., other Brassica species. Depending upon the oilseed and on the extraction process used, residual oil may be in the range of 2 to 12% by weight.

Gluten and Hominy Feed:

These are byproducts from corn processing. Corn gluten is a by-product of wet milling process to make cornstarch. Corn germ meal is golden-yellow and is mainly gluten, the high-protein portion of the corn kernel. Corn gluten meal may contain about 20% protein, about 2% fat and about 9% fiber. Corn gluten feed is an intermediate protein product that is rich in highly digestible fiber, and may contain about 21% protein, about 2.5% fat and about 8% fiber, but the crude protein values have ranged from 17 to 26% and the fat content may range from 1 to 7%. Wet corn gluten feed is similar but is not dried.

Brewers and Distillers Grains:

Brewers and distillers' dried grains are grain-based byproducts from the production of alcohol for a variety of uses, including alcohol-containing drinks such as beer and whiskey, petroleum additive and for other uses. Distillers grains contain the nutrients remaining after the corn starch is fermented into alcohol. The distillers grains can be wet or dried. Wet distillers grains are higher in protein and energy than corn gluten feed because gluten and oil remain in distillers grains. When distillers grains are dried they lose some energy value compared to wet products. Dried distillers grains and dried distillers grains with solubles are marketed widely around the world as a feed commodity. Dried brewers grains is the dried extract residue of barley malt alone or in mixture with other cereal grains from the manufacture of wort or beer. Distillers dried grains (DDG) are obtained after the removal of ethyl alcohol by distillation from the yeast fermentation of a grain or a grain mixture by separating the resultant coarse grain faction of the whole stillage and drying it by various methods. Distillers dried grains/solubles (DDGS) are recovered in the distillery and contain substantially all of the nutrients from the incoming corn except for the starch, which has been fermented into alcohol. It has been estimated that DDGS has at least threefold the nutrients as the incoming grain. DDGS has been estimated to contain about 27% protein, about 11% fat and about 9% fiber. Condensed distillers solubles (CDS) is a term generally used to refer to the evaporated co-products of the grain fermentation industry. On a dry matter basis CDS typically is about 29% protein, about 9% fat and about 4% fiber. The solubles are an excellent source of vitamins and minerals, including phosphorus and potassium. CDS can be dried to 5% moisture and marketed, but generally the dry matter content is between 25-50%. Wet distillers grains (WDG) can be used as livestock feed or dried into distillers grains (DDG). If syrup is added to wet distillers grains and dried, the resulting product is referred to as distillers dried gains with solubles (DDGS).

Wheat Millfeed Byproducts:

Byproducts such as shorts, millrun, bran and middlings are produced when wheat is processed to obtain certain food qualities. These byproducts are not desired for human consumption and so can be fed to animals. These byproducts of milling wheat for flour include varying amounts of bran, germ and flour. They are highly palatable, low in calcium and tend to be higher in phosphorus than most other grains and processed grain byproducts. Wheat bran is highest in fiber and phosphorus and lowest in energy. Wheat middlings (also called midds) is a common ingredient in cattle feeds. Midds are a by-product of the flour milling industry comprising several grades of granular particles containing different proportions of endosperm, bran and germ. Midds have about 96 percent of the energy value of barley and about 91 percent of the energy value of corn. Midds are palatable feedstuffs and can be included in a grain mixture at high levels.

Dairy Byproducts:

When milk is treated to form certain products such as dried skim milk, dried buttermilk, whole whey or whey protein concentrate a common significant step would be the removal of the butterfat.

Oats, Rice Byproducts:

These products from oats or rice processing include rolled-oats, crimped oats, pulverized oats, reground oat feed, oats or rice mill feeds and rice hulls, rice screenings, rice fines and rice gluten.

Grain Byproducts:

Byproducts such as barley feed, feed wheat, corn, milo and ground grain screenings. Byproducts of these materials may be used or, as needed to supplement the other byproducts, these grain materials may be used as primary feed materials, rather than as byproducts, in combination with the agricultural raw material byproducts described herein.

Additional agricultural raw material byproducts may include hydrolyzed feather meal, liquid whey, meat meal, meat and bone meal, molasses, peanut skins, tallow, yellow grease and fish meal, as well as other byproducts and other additives that are known for use in animal feeds.

The animal feed ingredients may also include nutritional additives. Non-limiting examples of nutritional additives include protein, amino acids, fats, fibrous materials, growth promoters and enzymes. For example, enzymes to improve the efficiency of raw material digestion may be added, such as enzymes to assist in conversion of non-starch carbohydrates into a useable form. Non-limiting examples of enzymes suitable for use in the animal feed compositions include endo-1,4-beta xylanase, endo-1,4-beta glucanase, alpha-galactosidase, alpha-amylase and 3-phytase. An example of a growth promoter is potassium diformate. Non-limiting examples of vitamins include vitamin A, the B vitamins, vitamin C, vitamins D₂ and D₃, vitamin E, vitamin K, biotin, choline, folic acid, pantothenic acid and any other vitamins needed. Non-limiting examples of minerals include, for example, iron, copper, zinc, manganese, cobalt, iodine and selenium, as well as calcium and salt, and may also include elements such as molybdenum, nickel, fluorine, vanadium, tin and silicon. Non-limiting examples of sweeteners include feed grade molasses, sugar and sodium saccharin. It is noted that a supplemental sweetener may not be needed, since glycerin has a substantial sweetness of its own. Other known additives for animal feeds may be added as well, such as microorganisms (e.g., yeasts), ionophores, anthelmintics and anticoccidials. Non-limiting examples of stabilizers include agar-agar, alginates such as calcium, sodium or potassium alginate, gums such as gum arabic, gum ghatti, guar gum, locust bean gum and gum tragacanth. Non-limiting examples of fatty acids include mono-, di- and tri-glyceride esters of fatty acids, free fatty acids, soap stock from vegetable oil refining, methyl and/or ethyl or higher alcohol esters of fatty acids, fatty acid salts. Non-limiting examples of fats include the aforementioned tallow, and lard, butterfat, Neat's foot oil, cod-liver oil and vegetable oils. Non-limiting examples of fibrous materials include the aforementioned oilseed hulls, dried apple pectin and pomace, almond hulls, bagasse, dried bakery product, buckwheat hulls, ground or cut grass, straw or alfalfa, beet fiber, psyllium CFS and hydrolyzed roughage. The foregoing list is exemplary and is not intended to be limiting. Any other known additives for animal feed can be added to the composition of the present invention. Particular additives may be selected and used in a nutritionally effective amount based on the type of animal to which the animal feed compositions of the present invention are to be fed, and based on the various nutritional contents of the animal feed ingredients that are being used in the animal feed.

### II. Methods for Producing and Using Animal Feed Compositions

In certain aspects, the present invention relates to a method of preparing an animal feed composition that is resistant to feeding by birds, the method comprising: (a) determining fat content and starch content in at least two animal feed ingredients; (b) selecting one or more animal feed ingredients based on a parameter selected from the group consisting of: (i) a fat content greater than or equal to a predetermined value for fat content; and (ii) a starch content greater than or equal to a predetermined value for starch content; (c) forming a pellet comprising the animal feed ingredients having a fat content or a starch content greater than or equal to the predetermined value; and (d) mixing the pellet with one or more animal feed ingredients that have a fat content and a starch content less than the predetermined value, thereby preparing the animal feed composition, wherein the one or more animal feed ingredients that have a fat content and a starch content less than the predetermined value are not pelletized.

Methods for producing pellets for animal feed are known in the art and are described, for example, in U.S. Pat. No. 3,496,858, US 2006/0170128, US 20070172540, and US 2010/0330251, each of which is incorporated by reference herein in its entirety. For example, pellets for animal feed may be produced by mixing one or more animal feed ingredients with a binder such as glycerin and then compressing the mixture into pellet form. Heat, e.g. in the form of steam, may be added to assist in the pelletizing and to drive off excess moisture content from the pellets. In one example, a conveyor conducts the animal feed ingredients to a blender, and a stream of a binder such as glycerin is fed to the blender. A feeder conducts the blended product to a pelletizer. Typical suitable pelletizers include pelletizing extruders and pellet mills. Steam may be added to enhance the pelletizing process by increasing temperature in the pelletizer. The pellets discharged from the pelletizer are conducted to a cooler to cool the pelletized product to a temperature suitable for storage.

In another example of an extrusion pelletizing process, an animal feed ingredient mixture is prepared from a binder and the animal feed ingredients, including any optional ingredients as needed for a particular formulation. The animal feed ingredient/binder mixture is combined in a suitable mixer. In the mixer, water or steam, or both, can be added to the mixture if needed. The mixture in the mixer is then fed into an extruder. The extruder may be any suitable extruder such as a single or twin screw extruder. The extruder may include suitable heating and cooling sections. For example, the mixture may be passed through a cooking zone, in which the mixture is both subjected to mechanical shear and heated to a temperature up to about 130° C. Suitable extruders are known in the pelletizing art. As the glycerin-byproduct mixture exits the extruder, it is forced through a pellet-forming die. Any suitable die may be used, as long as it provides pellets of the desired consistency and size.

In some embodiments, the starch content of the pellet is at least 0.5%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95% or 100% w/w of the pellet. In some embodiments, the starch content of the pellet is less than 0.5%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95% or 100% w/w of the pellet. Any of these values may be used to define a range for the starch content of the pellet. For example, in some embodiments, the starch content of the pellet is 20%-50%, 30%-60% or 40%-70% w/w of the pellet. In some embodiments, the starch content of the pellet is determined based on the percent dry matter of the pellet.

Methods for determining starch content of animal feed ingredients are known in the art and are described, for example, in US 2009/0119800 (which is incorporated by reference in its entirety), and kits for determining starch content of animal feed ingredients are commercially available (e.g Megazyme's Total starch Assay Kit—AOAC Method 996.11). For example, in one embodiment, the animal feed ingredient is lyophilized and ground to fine powder and the starch is precipitated by ethanol precipitation. The starch is resuspended in water and treated with α-amylase and amyloglucosidase to digest the starch. GOPOD) reagent (containing >12000 U Glucose Oxidase, >650 U Peroxidase & 4-aminoantipyrine 80 mg in 1 liter water) is added to each tube for determination of D-glucose content. The OD is measured at 510 nm, and observed OD (over a reagent blank) for each sample compared to that for D-glucose control is used to calculate the amount of free glucose released by the hydrolytic reaction catalyzed by amylase and amyloglucosidase from the starch present in each sample.

In some embodiments, the fat content of the pellet is at least 0.5%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95% or 100% w/w of the pellet. In some embodiments, the fat content of the pellet is less than 0.5%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95% or 100% w/w of the pellet. Any of these values may be used to define a range for fat content of the pellet. For example, in some embodiments, the fat content of the pellet is 5%-10%, 5%-20% or 10%-20% w/w of the pellet. In some embodiments, the fat content of the pellet is determined based on the percent dry matter of the pellet. Methods of determining fat content of an animal feed pellet are known in the art and are described, for example, in Ru et al., 2000, Asian-Aus. J. Anim. Sci. 13(7): 1017-1025. For example, fat content of the animal feed pellet may be determined by near infrared spectroscopy (NIR).

Pelletization of the animal feed ingredients that are high in starch and fat is performed to reduce consumption of the animal feed composition by birds. In some embodiments, pelletization of the animal feed ingredients that are high in starch and fat reduces consumption of the animal feed composition by birds by at least 1%, 2%, 3%, 4%, 5%, 6%, 7%, 8%, 9%, 10%, 15%, 20%, 25%, 30%, 35%, 40%, 45%, 50%, 60%, 70%, 80%, 90%, or 100% of the animal feed composition (w/w) relative to an animal feed composition in which the animal feed ingredients high in starch and lipids are not pelletized.

In one embodiment, the animal feed compositions described herein may be used for feeding ruminant animals. Ruminant animals include, for example, cattle, sheep and goats. The animal feed compositions may also be used to feed other animals, such as, for example, hogs or poultry.

The methods and compositions of the invention are effective for preventing a variety of birds from consuming an animal feed composition. For example, the invention may be used for repelling wild birds, including but not limited to, blackbird species (Icteridae), including red-winged blackbirds (Agelaius phoeniceus), grackles (Quiscalus spp.), yellow-headed blackbirds (Xanthocephalus xanthocephalus), and brown-headed cowbirds (Molothrus ater); starlings, including European starlings (Sturnus vulgaris); geese, including Canada geese (Branta canadensis), cackling geese (B. hutchinsii), and snow geese ((Chen caerulescens); crows, cranes, swans, pheasants, wild turkeys, pigeons, sparrows, woodpeckers, larks, robins, finches, and waxwings.

In certain aspects, the invention relates to a method of feeding an animal, comprising administering an animal feed composition as described herein to an animal. In certain embodiments, the animal feed composition administered to the animal comprises: (a) an animal feed pellet having a diameter greater than or equal to 9.5 mm; and

(b) one or more additional animal feed ingredients, wherein the animal feed pellet has a higher lipid content (w/w) and/or a higher starch content (w/w) than each of the one or more additional animal feed ingredients.

In certain aspects, the invention relates to a method of feeding an animal, comprising administering an animal feed pellet as described herein to an animal. In certain embodiments, the animal feed pellet comprises at least 5% w/w of fat and at least 30% w/w of starch and has a diameter greater than or equal to 9.5 mm.

As discussed above, consumption of animal feeds by birds may be reduced by incorporating the animal feed ingredients with high starch and/or fat content into a pellet that is of sufficient size to deter bird feeding. In certain embodiments, consumption of the animal feed by birds is reduced by at least 1%, 2%, 3%, 4%, 5%, 10%, 20%, 30%, 40%, 50%, 60%, 70%, 80%, 90%, or 95% relative to a conventional animal feed, for example, an animal feed in which the animal feed ingredients with high starch and/or fat content are not pelletized.

In certain aspects, the invention relates to a method of increasing weight gain in an animal, comprising administering an animal feed composition or an animal feed pellet as described herein to an animal. Administering an animal feed composition or animal feed pellet of the invention to an animal may increase weight gain in an animal by at least 1%, 2%, 3%, 4%, 5%, 10%, 20%, 30%, 40%, 50%, 60%, 70%, 80%, 90%, 100%, 200%, 300%, 400% or 500% relative to a conventional animal feed, for example, an animal feed in which the animal feed ingredients with high starch and/or fat content are not pelletized.

In certain aspects, the invention relates to a method of increasing milk production in a dairy cow, comprising administering an animal feed composition or animal feed pellet as described herein to a dairy cow. Administering an animal feed composition or animal feed pellet of the invention to a dairy cow may increase milk production in a dairy cow by at least 1%, 2%, 3%, 4%, 5%, 10%, 20%, 30%, 40%, 50%, 60%, 70%, 80%, 90% or 100% relative to a conventional animal feed, for example, an animal feed in which the animal feed ingredients with high starch and/or fat content are not pelletized.

In some embodiments of the methods described herein, the animal feed composition or the animal feed pellet is exposed to predation by birds during consumption of the feed by the animal, for example, a ruminant animal (e.g. a dairy cow), poultry or swine.

### III. Additional Components of the Animal Feed Compositions

In certain embodiments, the animal feed composition comprises further compounds in addition to the animal feed ingredients. For example, the animal feed composition may further comprise a bird repellant, a rodent repellant, a wildlife attractant (e.g. a bird attractant or a rodent attractant) and/or one or more therapeutic compounds for the treatment of disorders in the animal. In a particular embodiment, the animal feed composition further comprises a hyperimmunized egg product. Non-limiting examples of additional compounds that may be added to the animal feed composition are described below.

### A. Bird Repellants

The animal feed composition may further comprise a bird repellant. The bird repellant may be incorporated into the feed pellet, or into one or more additional feed ingredients that is not pelletized. In some embodiments, both the feed pellet and one or more additional feed ingredients comprise a bird repellant.

Bird repellent agents that are suitable for use in the invention are those that are efficacious as primary and/or secondary repellents. Primary repellents possess a quality (e.g., unpalatable taste, odor, irritation) that evokes reflexive withdrawal or escape behavior in an animal. In contrast, secondary repellents evoke an adverse physiological effect (e.g., illness, pain), which in turn is associated with a subsequently-avoided sensory stimulus (e.g., taste, odor, visual cue; Werner & Clark 2003, Understanding blackbird sensory systems and how repellent applications work. In: Linz, G. M., ed. Management of North American Blackbirds. Washington, D.C.: United States Department of Agriculture; pp. 31-40).

A variety of bird repellents have been previously described and are suitable for use herein, and include but are not limited to anthraquinones, flutolanil, anthranilates (including methyl and dimethyl anthranilate), methiocarb, caffeine, chlorpyrifos, (plus -cyhalothrin), methyl phenyl acetate, ethyl phenyl acetate, o-amino acerophenone, 2-amino-4,5-dimethyl ecetophenone, veratroyl amine, cinnamic aldehyde, cinnamic acid, cinnamide, and chitosan. These agents may be used alone or in combination. Similarly, the techniques for application of these agents are also well-known and have been described, including formulations, application rates, and application techniques. See, for example, Hermann (U.S. Pat. No. 3,941,887) describing the use of anthraquinones, Wilson (published U.S. application 2007/0178127 A1) describing the use of flutolanil, Kare (U.S. Pat. No. 2,967,128) and Mason (U.S. Pat. No. 4,790,990) describing the use of anthranilates and esters of phenyl acetic acid, Crocker and Perry (1990, ibid) describing the use of cinnamide, Schafer et al. (1983, ibid) and Werner et al. (2005, Caffeine Formulation for Avian Repellency. J Wildlife Management, 71:1676-1681) describing the use of caffeine and caffeine plus benzoate, respectively, and Preiser (U.S. Pat. No. 5,549,902) describing the use of any of anthranilates, methyl phenyl acetate, ethyl phenyl acetate, o-amino acerophenone, 2-amino-4,5-dimethyl ecetophenone, veratroyl amine, cinnamic aldehyde, cinnamic acid or cinnamide, the contents of each of which citations are incorporated by reference herein. Many formulations of these repellents are also available commercially, including but not limited to, 9,10-anthraquinone (AVIPEL, FLIGHT CONTROL PLUS, AV-1011, and AV-2022, all marketed by Arkion Life Sciences, New Castle, Del.), flutolanil (GWN-4770 and GWN-4771, marketed by the Gowan Company, Yuma, Ariz.), methyl anthranilate (BIRD SHIELD, marketed by the Bird Shield repellent Corp., Spokane, Wash.), methiocarb (MESUROL, marketed by the Gowan Company, Yuma, Ariz.), caffeine (Flavine North America, Inc., Closter, N.J.), and chlorpyrifos (plus -cyhalothrin; COBALT, marketed by Dow AgroSciences, Indianapolis, Ind.). Bird repellants are also described in WO2016/007179 and U.S. Pat. No. 6,328,986, each of which is incorporated by reference herein in its entirety.

The amount of the bird repellent agent used is selected to effectively repel birds from the animal feed composition. Thus, as used herein, an “effective amount” is defined as that amount which results in a significant repellence of the birds from the animal feed composition in comparison to an untreated control (i.e. an animal feed composition without repellent). The actual effective amount will vary with the particular repellent agent selected, its formulation, the bird pest, the target, and environmental factors, and may be readily determined by routine controlled experimentation. Suitable amounts and formulations are described in the prior art as noted hereinabove, and are also provided by the repellent manufacturers and suppliers. By way of example and without being limited thereto, preferred amounts of anthroquinone (AVIPEL, FLIGHT CONTROL PLUS, AV-1011 or AV-2022) are approximately 2,000 ppm active ingredient (a.i.) for most birds, but may be as low as 600 ppm a.i. for larks, preferred amounts of flutolanil are 35,000 ppm (GWN-4770) or 15,000 ppm (GWN-4771), preferred amounts of anthranilate (BIRD SHIELD) are 80,000 ppm a.i., preferred amounts of methiocarb (MESUROL 75-W) vary from 1,250 ppm a.i. for blackbirds to 30 ppm a.i. for larks and 15 ppm a.i. for robins, starlings, grackles, finches, and waxwings, preferred amounts of caffeine (1:1 caffeine plus sodium benzoate) are 3,500 ppm a.i., and preferred amounts of chlorpyrifos plus □-cyhalothrin (COBALT) are 2,500 ppm a.i.

### B. Rodents Repellants

Animal feed compositions of the invention may further comprise a rodent repellent. The rodent repellant may be incorporated into the feed pellet, or into one or more additional feed ingredients that is not pelletized. In some embodiments, both the feed pellet and one or more additional feed ingredients comprise a rodent repellant. The animal feed composition may comprise both a rodent repellant and a bird repellant as described above.

Rodent repellent agents which are suitable for use in the present disclosure include but are not limited to anthraquinones, flutolanil, anthranilates, methiocarb, caffeine, chlorpyrifos, cyhalothrin, methyl phenyl acetate, ethyl phenyl acetate, o-amino acerophenone, 2-amino-4,5-dimethyl ecetophenone, veratroyl amine, cinnamic aldehyde, cinnamic acid, cinnamide, allyl isothiocyanate, capsaicin, TRPV1, denatonium benzoate, quebracho, sucrose octaacetate, quinine, quinine hydrochloride, magnesium sulfate, o-aminoacetophenone, emetine dihydrochloride, aluminum ammonium sulphate, putrescent and volatile animal products (e.g. eggs, urine, blood meal, castor oil), putrescent and volatile plant products (e.g. pine needle oil, garlic oil, sinigrin), d-pulegone, thiram, glucosinolate, polygodial, piperine (e.g. Zanthoxylum piperitum), and combinations thereof.

The amount of the rodent repellent agent used is selected to effectively repel rodents from the animal feed composition. Thus, as used herein, an “effective amount” is defined as that amount which results in a significant repellence of rodents from the animal feed composition in comparison to an untreated control (i.e. an animal feed composition without repellent). The actual effective amount will vary with the particular repellent agent selected, its formulation, the rodent pest, the target, and environmental factors, and may be readily determined by routine controlled experimentation.

Effective amounts of repellent agents can be 100, 150, 200, 250, 300, 350, 400, 450, 500, 550, 600, 650, 700, 750, 800, 850, 900, 950, 1000, 1500, 2000, 2500, 3000, 3500, 4000, 4500, 5000, 5500, 6000, 6500, 7000, 7500, 8000, 8500, 9000, 9500, 10,000, 15,000, 20,000, 25,000, 30,000, 35,000, 40,000, 45,000, or 50,000 ppm. These values can be used to define a range, such as effective amounts in the range of 5000 to 20,000 ppm, or 1000 to 7500 ppm of repellent or attractant. The effective amount can be readily determined by routine controlled experimentation. By way of example and without being limited thereto, amounts of anthraquinone (AVIPEL SHIELD, FLIGHT CONTROL PLUS, AV-1011, AV-2022 or AV-4044) may be approximately 1-2% active ingredient (wt/wt) for most rodents, but may be as low as 0.01% active ingredient (wt/wt).

### C. Therapeutic Agents:

In certain embodiments, the animal feed composition further comprises a therapeutic agent. In some embodiments the therapeutic agent can be an antibiotic or hormone. Non-limiting examples of antibiotics include flavophospholipol, salinomycin sodium, avilamycin, robenidine HCl, lasalocid A sodium, halofuginone HBr, maduramicin ammonium alpha, narasin and diclazuril. Additional growth-promoting antibiotics include, for example, bacitracins, bambermycins, chlortetracycline, lincomycin penicillin, tylosin and virginiamycin. A non-limiting example of a hormone includes melengestrol acetate (an estrus suppressor). In a certain embodiment, the therapeutic agent can be an antibody, for example, an IgG or IgY antibody.

### D. Wildlife Attractants

The animal feed composition may further comprise a wildlife attractant, for example a bird attractant and/or a rodent attractant. Wildlife attractant agents which are suitable for use in the present disclosure include but are not limited to food-based agents (e.g. grains and grain products, seeds and seed products, nuts and nut products, nut butter, fruit and fruit products, dairy products, confectionery ingredients), plant fats, animal fats, protein, and combinations thereof.

### EXAMPLES

The following examples are intended to further illustrate the invention, but are not intended to limit the invention in any manner whatsoever.

### Example 1: Dairy Cattle Feed Component Preferences for Red-winged Blackbirds

Total mixed rations (TMR) for milk producing dairy cattle were analyzed in controlled feeding experiments with Red-winged blackbirds to determine which components of the animal feed the birds preferred.

This study was conducted at a commercial dairy in Northern Colorado, US. In 2015 the commercial dairy experienced approximately 10,000 to 25,000 birds per day between Nov. 1 and Mar. 31, 2015. The dairy had a herd size of 4,770 cows and 2,626 of these cows were in production. The herd consisted of 80% Holstein and 20% Holstein×Jersey cross. Milk cows on average were 40 months old and weighed 680 kg. Cattle days in milk were approximately 200. Milk production per head per day was approximately 37.1 kg, and bulk tank milk contained 3.50% milk fat and 3.08% milk protein.

Red-winged blackbirds (N=105) were live trapped from the commercial dairy using mist nets and modified Swedish crow traps. All blackbirds were transported to the United States Department of Agriculture, National Wildlife Research Center (NWRC) in Fort Collins, Colo. for feeding experiments. Blackbirds were quarantined for 2 weeks with ad libitum access to feed (Layena® pellets, Purina Animal Nutrition LLC., Saint Louis. Mo.) and water. Blackbirds were maintained in their quarantine cages until pre-test (4.9 m (L)×2.4 m (W)×2.4 m (H)). On Jan. 27, 2015, blackbirds were moved and housed five birds per 3.05 m (L)×3.05 m (W)×2.4 m (H) cage for pre-test and nutritional experiments. The pre-test lasted for 5 days. During pre-test, starlings were fed 1 kg of the TMR test diet, daily. The TMR offered was a high energy lactation ration fed to milk producing cows (30-200 days in milk (DIM)). The feeding experiment was conducted Feb. 3-7, 2015. Following the experiment, all captured blackbirds were euthanized following methods conforming to agency policy as stated in USDA/APHIS/WS Directive 2.505 and approved by the NWRC Internal Animal Care and Use Committee (QA-2369, J. C. Carlson—Study Director).

### Red-Winged Blackbird Cage Trial

Estimating Cattle Total Mixed Ration Component Preference by Red-winged Blackbirds.

For the purpose of estimating TMR component selection, a preference test was conducted for four consecutive days. On each of the test days red-winged blackbirds within 10 cages (n=° ° 10; 5 birds per cage) were offered eight TMR components likely to be consumed by red-winged blackbirds; steamed flaked corn (SFC), PROPEL® energy nugget (EN), canola meal (CM), cracked corn (CC), ground corn (GC), soy bean meal (SBM), cotton seed (CS), and lactating mineral (LM). The PROPEL® energy nugget is a pellet approximately 6 mm in diameter and containing ≥10% crude protein; ≥50% crude fat; and ≤53.5% crude fiber. The eight components were offered to red-winged blackbirds in each cage within separate oil pans (0.304 m (W)×0.1016 m (D)). Within each oil pan red-winged blackbirds were offered 100 g of each individual ration component. An additional cage absent of blackbirds contained 100 g of each component for estimates of daily feed desiccation (i.e. evaporative water loss). Consumption was measured for each component using the following equation:

Consumption=(1−(Treatment/Dessication))×100.

Treatment refers to the grams of feed remaining after exposure to birds, and desiccation refers to the grams of feed remaining after exposure to air. Estimates of feed consumption were averaged between days for each component and reported as percent consumed.

Estimating Nutritional Offsets Caused by Red-Winged Blackbirds.

A high energy total mixed rations (TMR) was used for nutritional testing of blackbirds. Each morning at 6:00 a.m., 15 kg of high energy TMR provided to lactating cows (30 to 200 DIM) was collected directly from feed trucks at the commercial dairy and brought to NWRC-Fort Collins for nutritional testing. The TMR was offered daily within each of 10 cages (n=10; 5 blackbirds per cage). For four consecutive days, 1.1 kg of feed was weighed out for each cage. A total of 1 kg was offered to blackbirds within an aluminum tray (0.9 m (L)×0.6 m (W)×2.54 cm (D)) and the remaining 100 grams was used as a control sample. The control sample was placed outside the cage in a paper bag. Both control and blackbird consumed rations were identified by cage number and day. An additional 1 kg TMR sample was placed in a cage absent of blackbirds to estimate daily feed desiccation. Following 24 h of blackbird foraging, the consumed and desiccation samples were weighed. The blackbird consumed and control samples were then placed in a drying over for 24 h. After drying was complete, all samples were ground using a Model 4. Thomas® Wiley Mill (Thomas Scientific, Swedesboro, N.J. 08085). The Wiley Mill was opened and brushed clean after processing each individual sample to eliminate cross contamination. Ground samples were stored in a walk-in cooler until all samples were processed.

### Data Analysis

Nutritional Analysis.

Nutritional testing of dairy TMR samples was conducted at Cumberland Valley Analytical Services (CVAS, Hagerstown, Mich.) 21742) using near infrared reflectance spectroscopy (NIR) and conventional macronutrient assays. Near infrared reflectance spectroscopy was used to estimate: dry matter, moisture as a percentage of as-fed, crude protein, calcium, phosphorus, magnesium, acid detergent fiber, neutral detergent fiber, starch, crude fat, ash, sodium, total digestible nutrients, net energy lactation (Mcal/kg), net energy maintenance (Mcal/kg), net energy gain (Mcal/kg) and non-fiber carbohydrates on a dry matter basis. Wet-chemistry was used to determine: calcium, phosphorus, magnesium, potassium, iron (mg/kg), zinc (mg/kg) and copper (mg/kg).

Statistical Analysis.

Individual blackbird consumption of the TMR was estimated. To estimate grams of TMR consumed by individual birds, the difference in grams of TMR recovered within the desiccation versus blackbird exposed cages was assumed to reflect food consumed by birds. After accounting for desiccation we divided by the number of birds within each cage for the estimation of per-bird consumption:

TMR Consumption=(Desiccation−Treatment)/5

All nutrient composition data were analyzed using analysis of variance within mixed linear models, Proc Mixed, SAS 9.2. Fixed effects included treatment status (blackbird consumed and control rations) and cage was included as a random effect. Denominator degrees of freedom were calculated using the Satterthwaite approximation. All nutritional data were compared for blackbird consumed and control rations. A total of 22 univariable models (m=22) were created; one model for each of the nutritional variables reported by CVAS. Because multiple hypotheses were tested, we controlled for false discoveries using the Benjamini Hochberg procedure (Benjamini and Hochberg, 1995, J. R. Statist. Soc. 57: 289-300). For all analyses, the false discovery rate was set at α=0.05. Univariate analyses were ranked by p-value from smallest (1) to largest (m). Cutoff values for the rejection of null hypotheses were calculated as (rank/m)*α (See Table 1 below).

NRC Dairy Production Modeling.

Parameterization of the NRC dairy production model (2001) was based upon animal condition and feed formulation data provided by the commercial dairy, nutrition data provided CVAS and component selection data collected at NWRC-Fort Collins. The NRC model was paramaterized based upon the observed changes to % dry matter starch concentrations in blackbird consumed TMR relative to control TMR. We assumed that nutritional changes were influenced by blackbirds selectively consuming steam flaked corn (SFC), PROPELS energy nugget (EN), cracked corn within the corn silage (CC) and ground corn (GC). These assumptions were based upon red-winged blackbird consumption patterns observed in preference tests. These four components were selected because in preference experiments these components accounted for over 99% of all food consumed. In other words, if the birds ate 1 g or more of a component in the preference experiments, it was assumed that blackbirds would consume that component, in TMR, when found.

Using the nutrition data which quantified the starch concentration (% dry matter) between control (starch=31.13%, STDEV=0.49%) and starling exposed rations (starch=28.22%, STDEV=0.65%), the starch content of the four components consumed by blackbirds in the preference experiment (SFC=71%; EN=27.9%, corn silage=28.6%, GC=71.6%) and their DM percent of total TMR (SFC=13.8%; EN 1.7%; corn silage=29.5%; ground corn=12.6%), the amount of each component consumed can be estimated. These four components were selected because in preference experiments they accounted for over 99% of all food consumed. In other words, if the birds ate 2% or more of a component in the preference experiments we assumed blackbirds would consume that component, in TMR, when found. All other components were assumed to be unimpacted by bird feeding and the mass percent of the remaining components were increased by a constant scalar so that the resulting feed composition summed to 100%. These corrected feed component values were then used to parameterize the NRC dairy production model for blackbird consumed rations (See Table 2 below).

### Results

TABLE 1
Analysis of Red-winged blackbird exposed and control total mixed rations
(TMR) for milk producing dairy cattle.

| | | Blackbird | Control | | | Cutoff |
|---|---|---|---|---|---|---|
| Variable | Units | Mean (X) | Mean (X) | P-Value | Rank¹ | Value² |
| Net Energy Lactation | Mcal/kg | 0.350 | 0.379 | <0.0001 | 4.5 | 0.0102 |
| Net Energy Maintenance | Mcal/kg | 0.359 | 0.376 | <0.0001 | 4.5 | 0.0102 |
| Net Energy Gain | Mcal/kg | 0.231 | 0.247 | <0.0001 | 4.5 | 0.0102 |
| Starch | % DM | 28.221 | 31.128 | <0.0001 | 4.5 | 0.0102 |
| Crude Fat | % DM | 4.625 | 5.482 | <0.0001 | 4.5 | 0.0102 |
| Potassium | % DM | 1.252 | 1.159 | <0.0001 | 4.5 | 0.0102 |
| Total Digestible Nutrients | % DM | 73.459 | 76.184 | <0.0001 | 4.5 | 0.0102 |
| Non Fiber Carbohydrates | % DM | 40.25 | 42.634 | <0.0001 | 4.5 | 0.0102 |
| Neutral Detergent Fiber | % DM | 26.725 | 24.825 | 0.0005 | 9.0 | 0.0205 |
| Acid Detergent Fiber | % DM | 17.793 | 16.134 | 0.0017 | 10.5 | 0.0239 |
| Calcium | % DM | 1.283 | 1.179 | 0.0017 | 10.5 | 0.0239 |
| Ash | % DM | 8.195 | 7.724 | 0.002 | 12 | 0.0273 |
| Crude Protein | % DM | 20.203 | 19.347 | 0.0028 | 13 | 0.0295 |
| Phosphorus | % DM | 0.468 | 0.448 | 0.0031 | 14 | 0.0318 |
| Copper | PPM | 31.938 | 30.282 | 0.0073 | 15 | 0.0341 |
| Magnesium | % DM | 0.396 | 0.376 | 0.0317 | 16 | 0.0364 |
| Manganese | PPM | 142.373 | 136.125 | 0.0356 | 17 | 0.0386 |
| Zinc | PPM | 279.656 | 253.186 | 0.0691 | 18 | 0.0409 |
| Dry Matter | % DM | 96.543 | 96.409 | 0.1994 | 19.5 | 0.0443 |
| Moisture | % DM | 3.456 | 3.591 | 0.1994 | 19.5 | 0.0443 |
| Sodium | % DM | 0.451 | 0.444 | 0.4531 | 21 | 0.0477 |
| Iron | PPM | 361.531 | 361.218 | 0.9863 | 22 | 0.05 |

¹Rank order of p-values from analyses of nutritional cattle feed samples
²Benjamini Hochberg cutoff values for rejection of null hypotheses.

TABLE 2
Dairy total mixed rations (TMR) components for control
and red-winged blackbird consumed rations.

| | | Red-winged | |
|---|---|---|---|
| High Energy TMR | Control DM | blackbird | Percent |
| Components | (kg/cow) | DM (kg/cow) ¹ | Reduction² |
| Alfalfa Hay | 2.52 | 2.52 | 0 |
| Canola Meal | 1.65 | 1.65 | 0 |
| Corn Silage³ | 7.50 | 6.79 | 9.5% |
| Energy Nugget | 0.43 | 0.39 | 9.6% |
| Grass Hay | 0.73 | 0.73 | 0 |
| Ground corn | 3.22 | 2.46 | 23.6% |
| Lactating Mineral | 0.86 | 0.86 | 0 |
| Soybean Meal | 1.70 | 1.70 | 0 |
| Steam Flaked Corn | 3.52 | 2.65 | 24.7% |
| Wet Brewers Grain | 0.82 | 0.82 | 0 |
| Wet Distillers Grain | 0.80 | 0.80 | 0 |
| Whole Cottonseed | 1.71 | 1.71 | 0 |

DM is dry matter.
¹ Red-winged blackbird DM (kg/cow) refers to the amount of dry matter of feed, measured in kg, offered to a single cow per day.
²Percent reduction refers to our estimated change in red-winged blackbird consumed feed relative to the control rations for each specific component.
³Change in corn silage reflects the change based upon red-winged blackbirds preference for cracked corn within the ensiled feed.

### Estimating Component Selection and Preference by Red-Winged Blackbirds

Red-winged blackbirds primarily selected steam flaked corn among the eight components offered during component selection testing (FIG. 1). Among all foods consumed, various corn products (flaked, cracked and ground corn) accounted for 78% of all foods consumed by red-winged blackbirds.

### Nutritional Analysis

The nutritional data provided by CVAS suggests red-winged blackbird consumption significantly altered the nutritional characteristics of TMR (Table 1). Red-winged blackbird consumed rations had reduced Net Energy Lactation (NEL) (P<0.0001), Net Energy Maintenance (NEM) (P<0.0001), and Net Energy Gain (NEG) (P<0.0001) concentrations compared to the control rations. See Table 1. Red-winged blackbird consumed rations also had reduced concentrations of starch (P<0.0001), crude fat (P<0.0001) and total digestible nutrients (P<0.0001). Blackbird consumed rations had higher Acid Detergent Fiber (ADF) (P<0.0017). Neutral Detergent Fiber (NDF) (P<0.0005), potassium (P<0.0001) and calcium (P<0.0017) concentrations compared to the control rations. See Table 1.

### NRC Dairy Production Modeling

Based upon NRC (2001) dairy production model estimates, animal performance can be impacted as a consequence of blackbird consumption of TMR destined for lactating cows (Table 3). For Holsteins producing 37.1 kg of milk/d, total required net energy intake (NEI) was 35.8 Mcal/d. Within the control TMR, total required net energy intake (NEI) supplied was 41.2 Mcal/d and within the blackbird consumed TMR NEI supplied was 37.8 Mcal/d. The resulting energy balance for control and starling exposed rations was 5.4 and 2.0 Meal/day respectively. Consequently, Holsteins fed the control TMR were estimated to gain one condition score in 96 days and experience daily weight change due to reserves of 1.1 kg/day. Holsteins fed blackbird consumed TMR would gain one condition score in 254 days and experience daily weight change due to reserves of 0.4 kg/day. Accordingly, these results indicate that preventing predation of animal feed by birds would increase weight gain and milk production in dairy cows.

Significant differences in dairy TMR were observed between the Red-winged blackbird exposed and control rations. This data identifies metabolizable energy sources that need to be protected from blackbird feeding. The nutritional data for blackbird exposed and control rations enabled identification of various food sources that blackbirds would desire and would potentially consume, not just the components tested in the preference experiments. In other words, based upon an understanding of nutrient preference by blackbirds, it is possible to determine the desirability of ingredients to birds and whether or not alternatives should be used.

Red-winged blackbirds seem to be deliberately selecting components with high starch and high fat concentrations. Blackbirds deplete starch and fat from dairy TMR, and these nutrients are depleted by the selective sourcing of SFC, EN, and cracked corn within the corn silage (CC) from TMR. This suggests that bird species may be attracted to feedlots and dairies because of specific and easy-to-obtain nutrients within expensive processed corn and fat supplements. Previous research has shown that European starlings could not consume ≥1.27 cm diameter extruded pellets (Depenbusch et al., 2011, Human-Wildlife Interactions 5:58-65). Yet, starlings provided 3/16″ (0.48 cm) diameter pig pellets consumed the rations at a rate more than eight times greater than granular hog meal (Twedt and Glahn, 1982, Proceedings 10^{th} Verterbrate Pest Conference, Monterey, Calif., pages 159-163); leading Glahn et al. (1983, Wild. Soc. Bull. 11: 366-372)) to surmise that starling consumption of food particles appears to be strongly influenced by feed form and size. This information is important because it appeared that blackbirds strongly preferred flaked corn over ground corn in our preference experiments, suggesting nutrient sourcing by blackbirds also appears to be influenced by feed form and size. If a starling cannot consume particles of ≥1.27 cm, the smaller red-winged blackbird will most likely not be able to consume the food as well. Altering the size of fat nugget and flaked corn, or combining highly desired components into approximately 1 cm diameter pellets or finely milled particles may be a cost-effective and non-lethal IPM strategy to manage bird damage at dairies.

TABLE 3
National Research Council, dairy production model output
for control and red-winged blackbird consumed rations.

| | | Blackbird |
|---|---|---|
| NRC model | Control | consumed |
| output | TMR | TMR |
| NEI Required | 35.8 | 35.8 |
| (Mcal/day) | | |
| NEI Supplied | 41.2 | 37.8 |
| (Mcal/day) | | |
| NEI Balance | 5.4 | 2.0 |
| (Mcal/day) | | |
| Days to gain | 96 | 255 |
| One BCS | | |
| Weight Change | 1.1 | 0.4 |
| (kg/day) | | |
| RDP Required | 2541 | 2312 |
| (g/day) | | |
| RDP Supplied | 2584 | 2524 |
| (g/day) | | |
| RDP Balance (g/day) | 42 | 212 |
| RUP Required | 1300 | 1395 |
| (g/day) | | |
| RUP Supplied | 1853 | 1689 |
| (g/day) | | |
| RUP Balance (g/day) | 552 | 294 |
| MP - Bacterial | 1383 | 1258 |
| (g/day) | | |
| MP - RUP | 1447 | 1305 |
| (g/day) | | |
| MP - Endogenous | 120 | 109 |
| (g/day) | | |

NEI is total required net energy intake; TMR is total mixed rations; BCS is the body condition score; RDP is rumen digestible protein: RUP is rumen undigestible protein; MP is metabolizable protein.

### Example 2: Dairy Cattle Feed Component Preferences for European Starling

This study was conducted at a commercial dairy in Northern Colorado. In 2012 the commercial dairy experienced approximately 5,000 to 15,000 starlings per day between Nov. 15 and Mar. 31, 2012. The dairy had a herd size of 2.767 cows and 1,403 of these cows were in production. The herd consisted of 80% Holstein and 20% Holstein×Jersey cross. Milk cows on average were 43 months old and weighed 590 kg. Cattle days in milk were approximately 200. Milk production per head per day for late lactation cattle (days in milk (DIM)≥168) was approximately 32 kg, and bulk tank milk contained 3.48% milk fat and 3.02% milk protein. Starlings (N=105) were live trapped from the commercial dairy using mist nets and modified Swedish crow traps. All starlings were transported to the United States Department of Agriculture, National Wildlife Research Center (NWRC) in Fort Collins, Colo. for feeding experiments. Starlings were quarantined for 2 weeks with ad libitum access to feed (LAYENA® pellets, Purina Animal Nutrition LLC., Saint Louis, Mo.) and water. Starlings were maintained in their quarantine cages until pre-test (4.9 m (L)×2.4 m (W)×2.4 m (H)). On Feb. 29, 2012 starlings were moved and housed five birds per 3.05 m (L)×3.05 m (W)×2.4 m (H) cage for pre-test and nutritional experiments. The pre-test lasted for 5 days. During pre-test, starlings were fed 1 kg of the TMR test diet, daily. The TMR offered was a late lactation ration fed to 75 milk producing cows. The feeding experiment was conducted from Mar. 5-8, 2012. Following the experiment, all captured starlings were euthanized following methods conforming to agency policy as stated in USDA/APHIS/WS Directive 2.505 and approved by the NWRC Internal Animal Care and Use Committee (QA-1742, J. C. Carlson—Study Director).

### European Starling Cage Trials

Estimating Cattle Total Mixed Ration Component Preference by European Starlings.

For the purpose of estimating feed component selection, a component selection test was conducted during four consecutive days. On each of test days 1 and 2, starlings within each of 10 cages (n=10; 5 starlings per cage) were offered one bowl that contained the TMR. The seven TMR components were steamed flaked corn (SFC), corn silage (CS), PROPELS energy nugget (EN), corn gluten (CG), dry distiller's grain (DDG), canola meal (CM), and mineral supplement. On each of test days 3 and 4, the same starlings within each of the 10 cages were offered one bowl that contained the TMR excluding the most favored component observed during test days 1 and 2 (i.e. EN). For the purpose of estimating feed component preference, one additional cage (5 starlings) was provided TMR separated into the seven ration components (i.e. 7 bowls each containing 100 g of individual ration components). The component preference test was conducted for two days. An additional cage absent of starlings contained 100 g of each component for estimates of daily feed desiccation (i.e. evaporative water loss).

Percent consumed was measured for each component using the following equation:

Consumption=(1−(treatment/desiccation))×100

Treatment refers to the grams of feed remaining after exposure to birds, and desiccation refers to the grams of feed remaining after exposure to air. Estimates of teed consumption were averaged between days for each of the selection and preference tests, and reported as percent consumed.

Estimating Nutritional Offsets Caused by European Starlings.

A high energy TMR was used for nutritional testing of starlings. Each morning at 6:00 a.m., 15 kg of late lactation TMR was collected directly from feed trucks at the commercial dairy and brought to NWRC-102 Fort Collins for nutritional testing. The TMR was offered daily within each of 10 cages (n=10; 5 starlings per cage). For four consecutive days, 1.1 kg of feed was weighed out for each cage. A total of 1 kg was offered to starlings within an aluminum tray (0.9 m (L)×0.6 m (W)×2.54 cm (D)) and the remaining 100 grams was used as a control sample. The control sample was placed outside the cage in a paper bag. Both control and starling consumed rations were identified by cage number and day. An additional 1 kg TMR sample was placed in a cage absent of starlings to estimate daily feed desiccation. Following 24 h of starling foraging, the starling consumed and desiccation samples were weighed. The starling consumed, control and desiccation samples were then placed in a drying oven for 24 h. After drying was complete, all samples were ground using a Model 4, Thomas® Wiley Mill (Thomas Scientific, Swedesboro, N.J. 08085). The Wiley Mill was opened and brushed clean after processing each individual sample to eliminate cross contamination. Ground samples were stored in a walk-in cooler until all samples were processed.

### Data Analysis

Nutritional Analysis.

Nutritional testing of dairy TMR samples was conducted at Cumberland Valley Analytical Services (CVAS, Hagerstown, Md. 21742) using near infrared reflectance spectroscopy (NIR) and wet-chemistry analysis. Near infrared reflectance spectroscopy was used to estimate: dry matter, moisture as a percentage of as-fed, crude protein, calcium, phosphorus, magnesium, acid detergent fiber, neutral detergent fiber, starch, crude fat, ash, sodium, total digestible nutrients, net energy lactation (Mcal/kg), net energy maintenance (Mcal/kg), net energy gain (Mcal/kg) and non-fiber carbohydrates on a dry matter basis. Wet-chemistry was used to determine: calcium, phosphorus, magnesium, potassium, iron (mg/kg), zinc (mg/kg) and copper (mg/kg).

Statistical Analysis.

Starling consumption of the seven TMR components is reported as percent consumed with standard deviations. To estimate grams of TMR consumed by cage and by bird we assumed the difference in grains of TMR recovered within the starling exposed and the desiccation cages reflects food consumed by starlings. This difference was then divided by the number of birds within each cage (5) to for the estimation of per-bird consumption:

$$TMR Consumption = \frac{( Desiccation - Treatment )}{5}$$

All nutrient composition data were analyzed using analysis of variance within mixed linear models, Proc Mixed, SAS 9.2. Fixed effects included treatment status (starling consumed and control rations) and cage was included as a random effect. Denominator degrees of freedom were calculated using the Satterthwaite approximation. All nutritional data were compared for starling consumed and control rations. A total of 22 univariable models (m=22) were created, one model for each of the nutritional variables reported by CVAS. Because multiple hypotheses were tested, false discoveries were controlled for using the Benjamini Hochberg procedure (Benjamini and Hochberg 1995). For all analyses, the false discovery rate was set at α=0.05. Univariate analyses were ranked by p-value from smallest (1) to largest (in). Cutoff values for the rejection of null hypotheses were calculated as (rank/m)*α (See Table 4 below).

NRC Dairy Production Modeling.

Parameterization of the NRC dairy production model (2001) was based upon animal condition and feed formulation data provided by the commercial dairy, nutrition data provided CVAS and component selection data collected at NWRC-Fort Collins. The NRC model was parameterized based upon the component selection data, with the assumption that nutritional changes were influenced by starlings selectively consuming energy nugget and steam flaked corn. Using the known change in crude fat between control and starling exposed rations, it was estimated that the amount of steam flaked corn (SFC) and energy nugget (EN) remaining following starling consumption using the linear equation below:

TMR % CF=X*(SFC % CF+EN % CF)+Σ% CF for all other TMR components

Where X=change in % CF attributed to starling consumption of EN and SFC, SFC % CF is percent of crude fat within the SFC component, EN % CF is the percent of crude fat within the EN component. All other components were assumed to be unimpacted by bird feeding and set to the relative mass distribution of the remaining feed components. To estimate the impact of starling foraging on feed formulation, the mass percent of the remaining components were increased by a constant scalar so that the resulting feed composition summed to 100%. The dry mass and wet mass for each feed component were calculated from these consumption corrected distributions. These corrected feed component values were then used to parameterize the NRC dairy production model for starling consumed rations (See Table 3 below).

### Results

### Estimating Component Selection and Preference by European Starlings

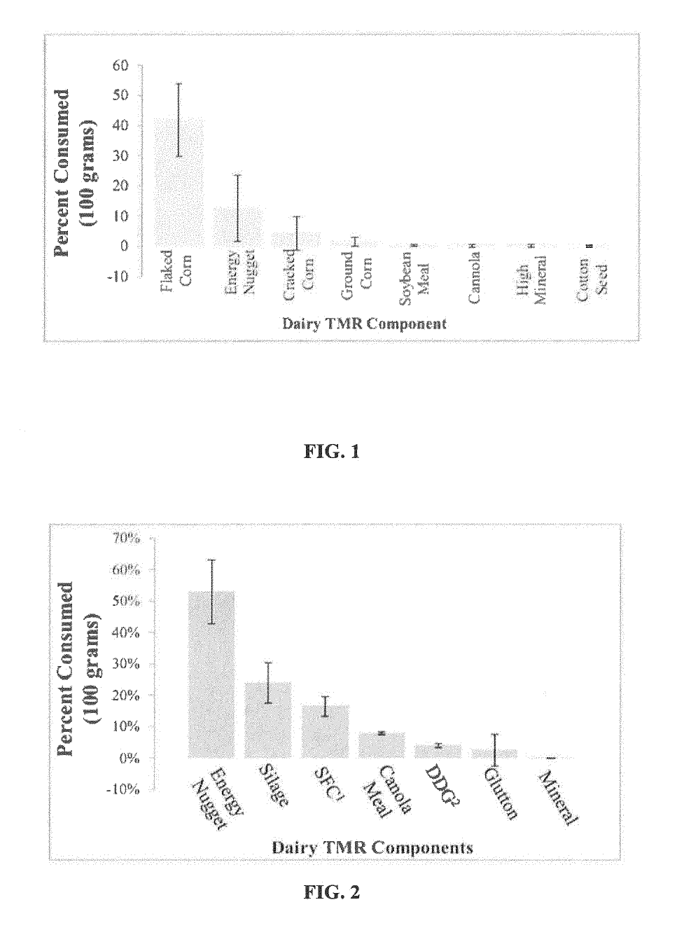

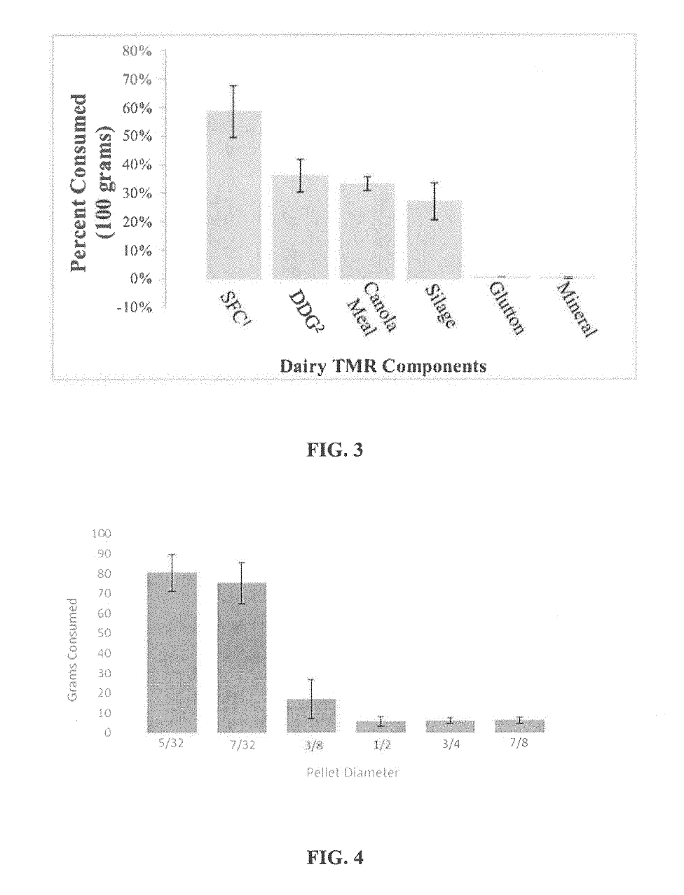

Starlings primarily selected the energy nugget (EN) among the seven components offered during component selection testing (FIG. 2). In the second selection test, excluding the EN, starlings primarily selected steamed flaked corn (SFC) (FIG. 3).

### Nutritional Analysis

The nutritional data suggests starling consumption significantly altered the nutritional characteristics of TMR (Table 4). Starling consumed rations had lower dry matter concentrations of net energy lactation (NEL) (P<0.0001), net energy maintenance (NEM) (P<0.0001), and net energy gain (NEG) (P<0.0001). Starling consumed rations also had lower dry matter concentrations of starch (P<0.0001), crude fat (P<0.0001), total digestible nutrients (P<0.0001), and crude protein (P=0.0378). Starling consumed rations had higher dry matter concentrations of acid detergent fiber (ADF) (P<0.0001), neutral detergent fiber (NDF) (P<0.0001), potassium (P<0.0001) and calcium (P<0.0001).

TABLE 4
Analysis of starling exposed and control total mixed rations (TMR) for milk producing
dairy cattle. DM is dry matter.

| | | Starling | Control | | | Cutoff |
|---|---|---|---|---|---|---|
| Variable | Metric | Mean | Mean | P-Value | Rank¹ | Value² |
| Net Energy Lactation | Mcal/kg | 1.61 | 1.72 | <0.0001 | 5.5 | 0.0125 |
| Net Energy Maintenance | Mcal/kg | 1.64 | 1.77 | <0.0001 | 5.5 | 0.0125 |
| Net Energy Gain | Mcal/kg | 1.03 | 1.15 | <0.0001 | 5.5 | 0.0125 |
| Acid Detergent Fiber | % DM | 22.107 | 18.985 | <0.0001 | 5.5 | 0.0125 |
| Neutral Detergent Fiber | % DM | 35.010 | 30.028 | <0.0001 | 5.5 | 0.0125 |
| Starch | % DM | 24.550 | 28.668 | <0.0001 | 5.5 | 0.0125 |
| Crude Fat | % DM | 3.295 | 4.359 | <0.0001 | 5.5 | 0.0125 |
| Potassium | % DM | 1.282 | 1.094 | <0.0001 | 5.5 | 0.0125 |
| Iron | PPM | 268.825 | 224.925 | <0.0001 | 5.5 | 0.0125 |
| Total Digestible Nutrients | % DM | 70.135 | 74.563 | <0.0001 | 5.5 | 0.0125 |
| Calcium | % DM | 0.841 | 0.687 | 0.0001 | 11.5 | 0.0261 |
| Magnesium | % DM | 0.376 | 0.342 | 0.0001 | 11.5 | 0.0261 |
| Dry Matter | % DM | 95.738 | 95.265 | 0.0003 | 14 | 0.0318 |
| Moisture | % DM | 4.263 | 4.738 | 0.0003 | 14 | 0.0318 |
| Ash | % DM | 7.491 | 6.861 | 0.0003 | 14 | 0.0318 |
| Non Fiber Carbohydrates | % DM | 39.478 | 43.368 | 0.0004 | 16 | 0.0364 |
| Zinc | PPM | 207.825 | 182.200 | 0.0027 | 17 | 0.0386 |
| Sodium | % DM | 0.489 | 0.438 | 0.0087 | 18 | 0.0409 |
| Manganese | PPM | 114.100 | 102.425 | 0.0097 | 19 | 0.0432 |
| Crude Protein | % DM | 15.788 | 16.360 | 0.0375 | 20 | 0.0455 |
| Phosphorus | % DM | 0.392 | 0.384 | 0.1376 | 21 | 0.0477 |
| Copper | PPM | 22.700 | 21.925 | 0.2473 | 22 | 0.05 |

¹Rank order of p-values from analyses of nutritional cattle feed samples
²Benjamini Hochberg cutoff values for rejection of null hypotheses.

TABLE 5
Dairy TMR components for control and starling consumed rations.

| | Control | | | | |
|---|---|---|---|---|---|
| High Energy TMR | wet weight | | Control DM | Starling | Percent |
| Components | (kg/cow) | % DM | (kg/cow) | DM (kg/cow) ¹ | Reduction² |
| Corn Silage | 5.26 | 34 | 1.79 | 1.79 | 0 |
| Alfalfa Haylage | 2.31 | 30 | 0.69 | 0.69 | 0 |
| Wet Brewers Grain | 1.41 | 21 | 0.30 | 0.30 | 0 |
| Alfalfa Hay | 4.38 | 90 | 3.94 | 3.93 | 0 |
| Hominy | 3.40 | 90 | 3.06 | 3.06 | 0 |
| Dry Distillers | 1.34 | 90 | 1.21 | 1.21 | 0 |
| Grain X | | | | | |
| Late Mineral | 0.84 | 98 | 0.82 | 0.82 | 0 |
| Straw | 0.48 | 90 | 0.43 | 0.43 | 0 |
| Energy Nugget | 0.45 | 97 | 0.44 | 0.30 | 47.19 |
| Canola Meal | 2.81 | 90 | 2.53 | 2.53 | 0 |
| Steam Flaked Corn | 2.72 | 90 | 2.45 | 1.70 | 44.50 |

DM is dry matter. TMR is total mixed rations.
¹ Starling DM (kg/cow) refers to the amount of dry matter of feed, measured in kg, offered to a single cow per day.
²Percent reduction refers is a measurement of the reduction detected in starling consumed feed relative to the control rations for each specific component

### NRC Dairy Production Modeling

Based upon NRC (2001) dairy production model estimates, animal performance can be impacted as a consequence of starling consumption of the late lactation TMR (Table 6). For Holsteins producing 32 kg of milk/day, total required net energy intake (NEI) was 31.5 Meal/day. Within the control TMR, NEI supplied was 29.3 Meal/day and within the starling consumed TMR NEI supplied was 27.7 Meal/day. The resulting energy balance for control and starling exposed rations was −2.2 and −3.9 Meal/day respectively. Consequently, Holsteins fed the control TMR were estimated to lose one body condition score in 161 days and experience daily weight change due to reserves of −0.4 kg/day. Body condition score (BCS) describes the relative fatness or body condition of a cow through the use of a nine-point scale. A body condition score 1 cow is extremely thin, and a BCS 9 cow is extremely fat and obese. Thus a loss of BCS is indicative of fat loss. Holsteins fed starling consumed TMR would lose one body condition score in 91 days and experience daily weight change due to reserves of −0.8 kg/day. Thus the Holsteins fed starling consumed TMR exhibited more rapid weight and fat loss than Holsteins fed the control TMR.

These results suggests that bird consumption of cattle feed by invasive European starlings causes direct economic losses and can reduce the productive capacity (i.e. milk production) of dairies through the nutritional depletion of dairy total mixed rations. Accordingly, these results suggest that prevention of bird predation of animal feed would increase weight gain and milk production.

TABLE 6
National Research Council, dairy production model
output for control and starling consumed rations.

| | | Starling |
|---|---|---|
| NRC model | Control | consumed |
| output | TMR | TMR |
| NEI Required | 31.5 | 31.5 |
| (Mcal/day) | | |
| NEI Supplied | 29.3 | 27.7 |
| (Mcal/day) | | |
| NEI Balance | −2.2 | −3.9 |
| (Mcal/day) | | |
| Days to Lose | 161 | 91 |
| One BCS | | |
| Weight Change | −0.4 | −0.8 |
| (kg/day) | | |
| RDP Required | 1778 | 1674 |
| (g/day) | | |
| RDP Supplied | 2141 | 2154 |
| (g/day) | | |
| RDP Balance (g/day) | 363 | 481 |
| RUP Required | 2016 | 2134 |
| (g/day) | | |
| RUP Supplied | 1526 | 1469 |
| (g/day) | | |
| RUP Balance (g/day) | −490 | −665 |
| MP - Bacterial | 967 | 911 |
| (g/day) | | |
| MP - RUP | 780 | 733 |
| (g/day) | | |
| MP - Endogenous | 84 | 79 |
| (g/day) | | |

NEI is total required net energy intake; TMR is total mixed rations; BCS is the body condition score; RDP is rumen digestible protein; RUP is rumen undigestible protein; MP is metabolizable protein.

### Example 3: European Starling Feed Consumption of Pelleted Poultry Feed

Previous research has suggested that European starlings cannot consume extruded pellets ≥12.7 mm in diameter (Depenbusch et al., 2011, cited above). In this study, on Day 1 European starlings (n=132) were removed from quarantine and housed 2 birds 139 per 1.83 m×0.914 m×0.914 m (L×W×H, respectively) cage for pre-test and particle size testing. Pre-test lasted for 5 days. During the pre-test all cages were offered 150 g of nutritionally complete poultry layer pellet of 0.396 cm diameter (Ranch-Way Feeds, Fort Collins, Colo.). Starting on Day 6 the layer pellet offered to starlings was collected and weighed. Weigh back occurred for 3 days. Birds were ranked based upon their three-day average consumption of pre-test pellets and then cages were assigned to 1 of 6 test pellets (22.2 mm, 19.1 mm, 12.7 mm, 9.5 mm, 5.5 mm, or 4.0 mm diameter pellet). Treatment assignments were stratified based upon ranked consumption data such that treatment groups were similarly populated with high-low consumers. The pellets were all produced by Ranch-Way Feeds using the same nutritionally complete poultry layer feed offered to birds during pretreatment. Starting on Day 9, starlings were offered 150 grams of the test diet consisting of 1 of the 6 different pellet diameters (n=11 cages per treatment). An additional cage housed desiccation samples of feed for each of the six treatments. The following day the remaining feed was collected and weighed. The response variable is pellet consumption per cage and it was measured in grams of feed consumed using the following equation: Consumption per Cage=(Desiccation−Treatment). At the completion of all European starling feeding experiments, captured starlings were euthanized.

Pelleted feed was effective at deterring starlings from consuming food (F_{5, 66}=316.88. P<0.001, Table 7). A pellet size of 9.5 mm or larger inhibited starling consumption of feed. Pelleted feed of 5.5 mm and 4.0 mm did not deter starlings from consuming food relative to their pre-treatment consumption rates (F_{1, 22}=1.80, P<0.1934). Thus, pellet size of 9.5 mm in diameter or larger reduced starling consumption of feed by ≥79%. See Table 7 below and FIG. 4.

TABLE 7
Assessment of pellet feed as a deterrent to starling
consumption of livestock feed supplies.

| Pellet | | Feed | Feed | Confidence | Bonferroni |
|---|---|---|---|---|---|
| Diameter | Sample | Offered | Consumed | Interval | Mean |
| (mm) | Size (n) | (g) | (g)¹ | 95%² | Difference³ |
| 22.2 | 11 | 150 | 6.25 | 2.22-10.29 | A |
| 19.1 | 11 | 150 | 6.08 | 2.05-10.12 | A |
| 12.7 | 11 | 150 | 5.62 | 1.58-9.65 | A |
| 9.5 | 11 | 150 | 16.82 | 12.78-20.85 | B |
| 5.5 | 11 | 150 | 75.14 | 71.10-79.17 | C |
| 3.9 | 11 | 150 | 80.45 | 76.42-84.49 | C |

¹Mean consumption/cage per treatment group. Each cage had 2 starlings.
²95% confidence intervals for the mean consumption/cage.
³Different letters identify non-overlapping confidence intervals based upon Bonferroni adjusted Least Squares Mean estimates.

1. An animal feed composition that is resistant to feeding by birds, the composition comprising:
(a) an animal feed pellet having a diameter greater than or equal to 9.5 mm; and
(b) one or more additional animal feed ingredients,
wherein the animal feed pellet has a higher lipid content (w/w) and/or a higher starch content (w/w) than each of the one or more additional animal feed ingredients.

2. The animal feed of claim 1, wherein the one or more additional animal feed ingredients have a diameter less than 9.5 mm.

3. The animal feed composition of claim 1, wherein the animal feed pellet comprises an animal feed ingredient selected from the group consisting of steam flaked corn, cracked corn and ground corn.

4. The animal feed composition of claim 1, wherein the animal feed pellet comprises at least 30% w/w of starch.

5. The animal feed composition of claim 1, wherein the animal feed pellet comprises at least 5% w/w of fat.

6. The animal feed composition of claim 1, wherein the composition further comprises an effective amount of a compound selected from the group consisting of a bird repellant, a rodent repellant, and a wildlife attractant.

7. The animal feed composition of claim 1, wherein the composition further comprises an antibody.

8. An animal feed pellet comprising at least 5% w/w of fat and at least 30% w/w of starch and having a diameter greater than or equal to 9.5 mm.

9. The animal feed pellet of claim 8, wherein the pellet comprises a total mixed ration (TMR).

10. The animal fled pellet of claim 8, wherein the pellet comprises an animal feed ingredient selected from the group consisting of steam flaked corn, cracked corn and ground corn.

11. A method of preparing an animal feed composition that is resistant to feeding by birds, the method comprising:
(a) determining fat content and starch content in at least two animal feed ingredients;
(b) selecting one or more animal feed ingredients based on a parameter selected from the group consisting of:
(i) a fat content greater than or equal to a predetermined value for fat content; and
(ii) a starch content greater than or equal to a predetermined value for starch content;
(c) forming an animal feed pellet comprising the animal feed ingredients having a fat content or a starch content greater than or equal to the predetermined value; and
(d) mixing the animal feed pellet with one or more animal feed ingredients that have a fat content and a starch content less than the predetermined value, thereby preparing the animal feed composition,
wherein the one or more animal feed ingredients that have a fat content and a starch content less than the predetermined value are not pelletized.

12. The method of claim 11, wherein the predetermined value for starch content is 30% w/w.

13. The method of claim 11, wherein the predetermined value for fat content is 5% w/w.

14. The method of claim 11, wherein the animal feed pellet has a diameter greater than or equal to 9.5 mm.

15. The method of claim 11, wherein the animal feed pellet comprises an animal feed ingredient selected from the group consisting of steam flaked corn, cracked corn and ground corn.

16. A method of feeding an animal, comprising administering the animal feed composition of claim 1 to an animal.

17. A method of increasing weight gain in an animal, comprising administering the animal feed composition of claim 1 to an animal.

18. A method of increasing milk production in a dairy cow, comprising administering the animal feed composition of claim 1 to a dairy cow.

19. The method of claim 17, wherein the animal feed composition is exposed to predation by birds during consumption of the feed by the animal.

20. The method of claim 17, wherein the animal is a ruminant animal, poultry or swine.
